SEQUENCE LISTING (7 sequences)

SEQ ID NO: 1 - protein, 13 aa, Homo sapiens
GRKKRRQRRRPPQ

SEQ ID NO: 2 - protein, 505 aa, Homo sapiens
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSGLVSSISRVLRIKFGKKEEEDEADKKEDDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPAAPSNHMNPVSNGLSPQVMSILGNPSAVPPQPQADFSISPLHGGLDSATSISASCSQRADSIKPGDSLPTSQAYCPPTYSTTGYSVDPVAGYQYGQYGQTAVDYLAKNVSLSTQRRMKLGEHSAVLGLLPVETGQAY

SEQ ID NO: 3 - protein, 224 aa, Balaenoptera acutorostrata
MELYETSPYFYQEPHFYDGENYLPVHLQGFEPPGYERTELSLSPEARVPLEDKGLGTPEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPGVPSECSSHSASCSPEWGSALEFGPNPGDHLLTADPTDAHNLHSLTSIVDSITVEDVAVAFPDETMPN

SEQ ID NO: 4 - protein, 319 aa, Sus scrofa
MELLSPPLRDVDLTGPDGSLCNFATADDFYDDPCFDSPDLRFFEDLDPRLVHVGALLKPEEHSHFPAAAHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYDGTYYSEAPSEPRPGKNAAVSSLDCLSSIVESISTESPAAPALLLADTPRESSPGPQEAAAGSEVERGTPTPSPDAAPQCPASANPNPIYQVL

SEQ ID NO: 5 - protein, 135 aa, Sus scrofa
GDFRCRCPAGFMDKTCSRPVSTCANEPCLNGGTCLQHSQVRFECLCKPKFTGPLCGRKRAAGPQQVTRLPSSYGLTYRLTPGVHELPVPQPEHRILKVSMKELTKSTPLLSEGQAICFTILGVLTSLVVLGTMGI

SEQ ID NO: 6 - protein, 504 aa, Phacochoerus africanus
MGETLGDSLIDPESDAFDTLSANISQEVTMVDTEMPFWPTNFGISSVDLSVMDDHSHSFDIKPFTTVDFSSISTPHYEDIPFPRADPMVADYKYDLKLQDYQSAIKVEPVSPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKNIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 7 - protein, 358 aa, Homo sapiens
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

(19) United States
(12) Patent Application Publication
(10) Pub. No.: US 2024/0002806 A1
(43) Pub. Date: Jan. 4, 2024

(54) METHOD FOR DIFFERENTIATING ADULT STEM CELLS INTO FINAL TISSUE

(71) Applicant: Innocent Meat GMBH, Papendorf (DE)

(72) Inventor: Patrick Nonnenmacher, Rostock (DE)

(73) Assignee: Innocent Meat GMBH, Papendorf (DE)

(21) Appl. No.: 18/119,314

(22) Filed: Mar. 9, 2023

Related U.S. Application Data
(60) Provisional application No. 63/318,402, filed on Mar. 10, 2022.

(51) Int. Cl. C12N 5/077

(52) U.S. Cl. CPC C12N 5/0658; C12N 5/0653; C12N 2506/45; C12N 2501/998

(57) ABSTRACT

A method for differentiating adult stem cells into a final tissue is described. The method utilizes adult stem cells as a primary material for cultivation of fat and muscle tissue, resulting in an easier induction potential to differentiate into the target cells. To induce the differentiation, transcription factors are used.

### CROSS-REFERENCE TO RELATED APPLICATIONS

This application claims priority under 35 USC § 119(e) to U.S. Provisional Application No. 63/318,402, filed Mar. 10, 2022, the entire contents of which is incorporated by reference in its entirety.

### SEQUENCE LISTING INCORPORATION BY REFERENCE

The contents of the electronic sequence listing (036271MG07UTL1US_sequence_listing.xml; Size: 9163 bytes; and Date of Creation: Jul. 24, 2023) is herein incorporated by reference in its entirety.

### FIELD OF THE INVENTION

The field of the invention and its embodiments relate to a novel method for differentiating adult stem cells into final tissue. More specifically, the field of the invention and its embodiments relate to a method that utilizes adult stem cells as a primary material for cultivation of fat and muscle tissue, resulting in an easier induction potential to differentiate into the target cells. As described herein, to induce the differentiation, transcription factors are used.

### BACKGROUND OF THE INVENTION

Adult stem cells are undifferentiated cells found throughout the body that divide to replenish dying cells and regenerate damaged tissues. Adult stem cells have the ability to differentiate into more than one cell type. Current methods to initiate differentiation of adult stem cells include adding unspecified peptides to media and/or altering the genetic information of target genes. However, these current methods have several drawbacks.

Thus, what is needed is an enhanced method for differentiating adult stem cells into a final tissue that does not require stable alteration of genetic information or alteration of gene expression. Moreover, what is needed is an enhanced method that utilizes adult stem cells as a primary material for cultivation of fat and muscle tissue, resulting in an easier induction potential to differentiate into the target cells. As described herein, to induce differentiation, transcription factors are used.

Examples of related art include:

CN110845625A describes a cell-penetrating peptide-pre-B cell leukemia transcription factor 1 fusion protein and preparation method and application thereof.

U.S. Ser. No. 10/525,100B2 describes activating transcription factor 5 (ATF5) peptides having a truncated ATF5 leucine zipper region and, optionally, a cell-penetrating region.

WO2019016795A1 describes a method for producing an edible composition, comprising incubating a three-dimensional porous scaffold and a plurality of cell types comprising: myoblasts or progenitor cells thereof, at least one type of extracellular (ECM)-secreting cell and endothelial cells or progenitor cells thereof, and inducing myoblasts differentiation into myotubes.

AU2016204474B2 describes engineered meat products formed as a plurality of at least partially fused layers. Each layer comprises at least partially fused multicellular bodies comprising non-human myocytes. The engineered meat is comestible.

U.S. Pat. No. 9,969,774B2 describes a cell-penetrating peptide and a composition for delivering a biologically active substance, a composition for gene therapy, and a method for gene therapy using the same.

WO2008086484A2 describes a fusion protein for treating cardiovascular disease that includes a transcription factor (TF) and a cell-penetrating peptide (CPP).

US20030143731A1 describes materials and methods involving the heterologous expression of transcription factors that are useful for effecting transcription of target genes in genetically engineered cells or organisms containing them.

One group describes a method of encoding human immunodeficiency virus type I (HIV-1) transactivator of transcription (TAT) by HIV-1. It has been found that HIV-1 TAT and its core peptide segment TAT47-57 play an important role in promoting the cellular uptake of coupled bioactive macromolecules, such as peptides, proteins, oligonucleotides, and drug molecules. HIV-1 TAT can also significantly increase the soluble expression of extrinsic proteins. However, the mechanism behind the cellular uptake of HIV-1 TAT-derived cell-penetrating peptide remains unclear (Zou, et al., 2017).

Another group describes that translocation through the plasma membrane is a major limiting step for the cellular delivery of macromolecules. A promising strategy to overcome this problem consists in the chemical conjugation (or fusion) to cell penetrating peptides (CPP) derived from proteins able to cross the plasma membrane. A large number of different cargo molecules such as oligonucleotides, peptides, peptide nucleic acids, proteins or even nanoparticles have been internalized in cells by this strategy. One of these translocating peptides was derived from the HIV-1 TAT protein (Silhol, et al., 2002).

Some similar systems exist in the art. However, their means of operation are substantially different from the present disclosure, as the other inventions fail to solve all the problems taught by the present disclosure.

### SUMMARY OF THE INVENTION

The present invention and its embodiments relate to a novel method for differentiating adult stem cells and/or induced pluripotent stem cells into a final tissue. In an embodiment, the present invention and its embodiments relate to a method that utilizes adult stem cells and/or induced pluripotent stem cells as a primary material for cultivation of fat and muscle tissue, resulting in an easier induction potential to differentiate into the target cells. As described herein, to induce the differentiation, transcription factors are used.

An embodiment of the present invention describes a method, which includes numerous process steps. For example, the method includes: utilizing a transcription factor to induce differentiation of adult stem cells (e.g., satellite cells and mesenchymal stem cells, IPSC (induced pluripotent stem cells)) into myocytes or adipocytes. The transcription factor may be a muscle-inducing protein/peptide or a fat-inducing protein/peptide. A non-exhaustive list of the muscle-inducing proteins/peptides include: PAX-7, Myf5, MyoD1 and Myogenin. A non-exhaustive list of the fat-inducing peptides include: Pref-1, PPAR-γ, and C/EBPα.

The method also includes applying the pre-myocytes or the pre-adipocytes to cell culture processing via addition to a culture medium. Next, the method includes: modifying the muscle-inducing protein/peptide or the fat-inducing protein/peptide with a cell penetrating peptide. In some examples, the cell penetrating peptide comprises a transactivator of transcription (TAT) signal peptide.

Further, when the muscle-inducing protein/peptide comprises MyoD1, the method may additionally include engaging in post-translational modification of MyoD1. In another example, when the fat-inducing protein/peptide comprises C/EBPα, the method further comprises: engaging in post-translational modification of the C/EBPα.

Moreover, in some implementations, the cell line is used to create a cultured, clean meat, or in vitro meat product.

### DETAILED DESCRIPTION OF THE INVENTION

The present invention and its embodiments relate to a novel method for differentiating adult stem cells into a final tissue. In an embodiment, the present invention relates to a method that utilizes adult stem cells as a primary material for cultivation of fat and muscle tissue, resulting in an easier induction potential to differentiate into the target cells. As described herein, to induce differentiation, transcription factors are used.

The process of transcription is the first stage of gene expression resulting in the production of a primary ribonucleic acid (RNA) transcript from deoxyribonucleic acid (DNA) of a particular gene. It therefore represents a critical first step in gene expression, which is followed by a number of post-transcriptional processes, such as RNA splicing and translation. These processes lead ultimately to the production of a functional protein.

Moreover, as well as this central role in gene expression in general, transcription also plays a part in specificity, since it is the primary target for the process of gene regulation, which results in different proteins being produced in different tissues. In fact, whilst some cases of regulation after transcription do exist, in most cases, selection occurs at this level by deciding which genes will be transcribed into the primary RNA transcript (Darnell, 1982). It is only following the gene transcription process that the other stages of gene expression, such as RNA splicing, occur automatically and result in the production of the corresponding protein.

Both basal transcription and its regulation are dependent upon specific protein factors, known as transcription factors. As described herein, a “transcription factor” is a protein that controls the rate and/or the amount of transcription of genetic information from DNA to messenger RNA, by binding to a specific DNA sequence. Specifically, transcription factors function to regulate genes in order to ensure they are expressed in the desired cells at the right time and in the right amount throughout the life of the cell and the organism. Transcription factors may work alone or with other proteins in a complex by promoting or blocking the recruitment of RNA polymerase to specific genes. Transcription factors are commonly classified into families on the basis of the precise protein structure which they use to mediate binding to DNA or to cause factor dimerization which is often essential for DNA binding. (Latchman, 1993).

The present invention describes use of selected transcription factors for the differentiation of satellite cells, mesenchymal stem cells and IPSC into myocytes and adipocytes. In an embodiment, their application to cell culture processes will be achieved via addition to cell culture media. To guarantee the correct uptake of transcription factors into the cell and finally into the nucleus, the peptides will be tagged with signal peptides that allow the entrance of these target-proteins into the cell. In an embodiment, the cell line may be used to create a cultured, clean meat, or in vitro meat product.

Thus, the present invention provides numerous benefits from similar methods and systems in the underlying technical field. For example, the present invention does not require the stable alteration of genetic information for alteration of gene expression. Accordingly, in one embodiment, the present invention relates to differentiation of stem cells in the absence of altering genes. Moreover, in an embodiment, the present invention couples and/or fuses transcription factors to a cell penetrating peptide to target the cell core for direct interaction with its DNA-Sequence. In a further embodiment, the present invention provides the possibility for heterologous expression of cell-penetrating-peptides coupled with transcription factors in genetically modified plants. Additionally, the present invention describes supplements to support the final differentiation into target tissue.

### HIV-1 Trans-Activator Gene (TAT)

The HIV-1 trans-activator gene (TAT) is essential for the latent transcription of the HIV virus, for its replication and for its gene expression (Karn, 2011). In addition, the TAT protein is able to conjugate with exogenous molecules (like nucleic acids, proteins, peptides or drug molecules), mediating their delivery into the cell through the plasma membrane or the nucleus through the nuclear membrane (Mae & Langel, 2006), (Milletti, 2012). The TAT protein belongs to the protein transduction domain (PTD) family. Its ability to fuse and deliver recombinant proteins, as well as therapeutic ones, has been studied in different fields (e.g., disease treatment and diagnosis), especially in research for cancer treatment (Wadia & Dowdy, 2005).

The HIV TAT protein transduction motif along with the Drosophila antennapedia (Antp) and the herpes simplex virus VP22 protein, are generally the most studied PTDs. They differ in their length and amino acid sequence, but they share the same “cell-penetrating effect” (Schwarze, Hruska, & Dowdy, 2000). PTDs are widely used because they transport macromolecules into cells, they have almost no cytotoxicity, and they typically do not modify the genome of the target cells. These characteristics give the PTDs an excellent biological safety profile. Their transduction abilities and characteristics interestingly appear to induce tissue-specific differentiation when the TAT protein is associated with transcription factors, e.g. in the myogenic differentiation (Patruno, M., et al., 2017) or in reprogramming adult stem cells.

The HIV-1 TAT protein is 86 amino acids long and consists of two exons: the first one comprising 72 amino acids and the second 14 (Green, M. & Loewenstein, P. M., 1988). According to its amino acid sequence, this protein can be divided into different domains, the most important one is the core domain (comprising aa 47-57) (Mann, D. A. & Frankel, A. D., 1991), (Vivès, E. Brodin, P. & Lebleu, B., 1997) which is at least partially responsible for the transducing ability of the TAT protein. The amino acid composition of the core domain is fundamental to carrying out the transduction of cargo (e.g., biomolecules) into target cells.

It should be understood that other HIV-1 TAT derived proteins can be used in conjunction with the present invention. The protein may comprise other subtype variants that are between 86 and 101 amino acids in length.

Some studies have shown that to enable efficient transmembrane movement, proteins and/or peptides that facilitate this movement must have at least a domain that is rich in basic amino acids (e.g., arginine or lysine). In fact, an enrichment in positively charged amino acids enhances its function (e.g., arginine, histidine, or lysine). This led researchers to improve the efficiency of the transduction of TAT by modifying its sequence adding basic amino acids or by modification of its structure (Ho, A., et al., 2001). Indeed, it has been demonstrated that short peptides rich in arginine are rapidly internalized by cells in a receptor-independent manner (Green, M. & Loewenstein, P. M., 1988), and as such, it was suggested that the process of TAT internalization occurs through adsorptive endocytosis. This does not happen for the TAT basic domain when fused to protein cargos (Fittipaldi, A. & Giacca, M., 2005).

The selected sequence described herein contains the following: GRKKRRQRRRPPQ (Gly-Arg-Lys-Lys-Arg-Arg-Gln-Arg-Arg-Arg-Pro-Pro-Gln). SEQ ID NO: 1

### Myoblast Determination Protein 1 (MyoD)

MyoD for example is a transcription factor that plays a key role in muscular differentiation (Davis, R. L., Weintraub, H. & Lassar, A. B., 1987). One group showed that the MyoD protein fused with TAT has a higher differentiating potential than the wild type MyoD (Hidema, S., et al., 2012). Recently, the potential of the complex TAT-MyoD in inducing equine peripheral blood mesenchymal stem cells (PB-MSCs) towards the myogenic fate was evaluated (Patruno, M., et al., 2017). Mesenchymal stem cells are an undifferentiated multipotent cell population which deserve particular attention because they offer an alternative therapeutic solution for muscle diseases (Uysal, C. & Mizuno, H., 2010). Up to now, scarce data were present in literature about the differentiation of MSCs into myoblasts, but in vitro, it has been shown that MSCs may differentiate into skeletal muscle cells with conditioned medium as well as in co-culture with a fusion between MSCs and myoblasts (Dezawa, M., et al., 2005). Although data indicates that TAT-MyoD induces myogenic differentiation in naturally predisposed cells only, like the C2C12 cell line (Noda, T., et al., 2009) or the mouse muscle primary cells, some authors demonstrated that the efficiency of myogenic differentiation with Tat-MyoD transduction of human adipose-derived stem cells was reached only when it was fused with C2C12 myoblasts (Sung, M. S., et al., 2013).

The above-mentioned group has discovered that the supplement of TAT-MyoD alone is not sufficient to induce cellular differentiation, even if it activated the myogenic pathway at the nuclear level. Therefore, in order to achieve the myogenic differentiation of MSCs, a conditioned medium was added in the present invention, which creates a suitable in vitro microenvironment for the differentiation towards muscle cells. These results suggest that TAT-mediated protein transduction system, if supported by a conditioned medium, represents a useful methodology to induce myoblasts differentiation. The conditioned medium describes thereby cell culture media that has been in contact with the secretome of supportive cells that produce growth factors and other important molecules and secrete it into the medium. Moreover, this indicates that the development of myogenic phenotypes of mesenchymal stem cells by TAT-MyoD construct depends on time and culture conditions, highlighting the essential role of the in vitro microenvironment in terms of secreted factors and cell contacts. Other studies have confirmed the necessity of having other factors apart from the genetic ones (e.g., MyoD) to commit undifferentiated cells (Kashanchi, F., et al., 1996).

The advantage of having a robust myogenic differentiation method by means of the TAT-mediated protein transduction consists in obtaining committed myogenic cells derived from an abundant cell source, like peripheral blood, without the need to fuse them with other cells. Certainly, this innovative approach of protein transduction with TAT fused with various transcription factors appears extremely interesting in the therapeutic and regenerative medicine field (Hua, W., et al., 2013).

### Differentiation in Muscle Tissue (Yin, H., Price, F., & Rudnicki, M. A., 2013)

In intact muscle, satellite cells are sublaminal and mitotically quiescent (Go phase). Quiescent satellite cells are characterized by their expression of Pax7, but not MyoD or Myogenin (Cornelison, D. D. & Wold, B. J., 1997). Upon exposure to signals from a damaged environment, satellite cells exit their quiescent state and start to proliferate (e.g., satellite cell activation). Proliferating satellite cells and their progeny are often referred to as “myogenic precursor cells (MPC)” or “adult myoblasts.” Unlike quiescent satellite cells, myogenic precursor cells are characterized by the rapid expression of myogenic transcription factors MyoD (Cornelison, D. D., et al., 2000), (Cornelison, D. D. & Wold, B. J., 1997), Myogenin (Füchtbauer, E. & Westphal, H., 1992), (Grounds, et al., 1992), (Smith, C. K., Janney, M. J., & Allen, R. E., 1994) and Myf5 (Cornelison, D. D. & Wold, B. J., 1997).

Of note, the presence of MyoD, desmin, and Myogenin in satellite cells was observed as early as 12 hours after injury, which is before any noticeable sign of satellite cell proliferation (Rantanen, J., et al., 1995). This early expression of MyoD is proposed to be associated with a subpopulation of committed satellite cells, which are poised to differentiate without proliferation (Rantanen, J., et al., 1995). In contrast, the majority of satellite cells express either MyoD or Myf5 by 24 hours following injury (Cornelison, D. D. & Wold, B. J., 1997) and subsequently co-express both factors by 48 hours (Cooper, R. N., et al., 1999). The ability of satellite cells to upregulate either MyoD or Myf5 suggests that these two transcription factors may have different functions in adult myogenesis.

A high ratio of Pax7 to MyoD (as seen in quiescent satellite cells) keeps satellite cells in their quiescent state. An intermediate ratio of Pax7 to MyoD allows satellite cells to proliferate, but not differentiate. Satellite cells with a low Pax7-to-MyoD ratio begin to differentiate, and further reduction in Pax7 levels are observed following activation of Myogenin.

Based on this and additional studies, the present invention tested different transcription factors in an attempt to regulate the state of muscle-progenitor-cells (Yin, H., Price, F., & Rudnicki, M. A., 2013).

### Differentiation in Fat Tissue

Preadipocyte factor 1 (Pref-1) is an EGF-repeat-containing transmembrane protein that inhibits adipogenesis. The extracellular domain of Pref-1 is cleaved by an TNF-α converting enzyme to generate the biologically active soluble form of Pref-1. The role of Pref-1 in adipogenesis has been firmly established by in vitro and in vivo studies. Pref-1 activates ERK/MAPK and upregulates Sox9 expression to inhibit adipocyte differentiation. Sox9 directly binds to the promoter regions of CCAAT/enhancer-binding protein-β and CCAAT/enhancer-binding protein-8 in order to suppress their promoter activities in preventing adipocyte differentiation (Wang, Y., Hudak, C., & Sul, H. S., 2010).

Adipogenic induction rapidly induces expression of the CAAT/enhancer-binding proteins (C/EBPs), C/EBPβ and C/EBPδ. These are key early regulators of adipogenesis, and the anti-adipogenic preadipocyte factor 1 (PREF1) has recently been shown to act through SOX9 in the direct regulation of the promoters for the genes encoding C/EBPβ and C/EBPδ (Wang, Y. & Sul, H. S., 2009). In addition, C/EBPβ appears to be the target of the proadipogenic desumoylating enzyme sentrin-specific peptidase 2 (SENP2) (Chung, S. S., et al., 2010). SENP2 is required to reduce the levels of C/EBPβ sumoylation, which would otherwise lead to its increased ubiquitylation and degradation of the protein.

Among the targets of C/EBPβ and C/EBPδ are the promoters of the genes encoding the key adipogenic transcription factors C/EBPα and PPARγ and the regulator of lipogenic genes for sterol-regulatory-element-binding protein 1 (SREBP1) (Rosen, E. D. & MacDougald, O. A., 2006), (White, U. A. & Stephens, J. M., 2010). PPARγ activates the promoter of the gene encoding C/EBPα and vice versa, creating a positive-feedback loop. In addition, PPARγ and C/EBPα induce the expression of genes that are involved in insulin sensitivity, lipogenesis, and lipolysis, including those encoding glucose transporter GLUT4 (also known as SLC2A4), fatty-acid-binding protein (FABP4, also known as adipocyte protein 2, aP2), lipoprotein lipase (LPL), sn-1-acylglycerol-3-phosphate acyltransferase 2 (AGPAT2), perilipin and the secreted factors adiponectin and leptin. Recent genome-wide binding analyses have revealed that PPARγ and C/EBPα cooperate on multiple binding sites in promoter regions, together regulating a wide range of genes expressed in developing and mature adipocytes (Lefterova, M. I., et al., 2008).

Based on this and further studies, the present invention tested different transcription factors to regulate the state of fat-progenitor-cells.

### Muscle-Influencing Peptides

### Paired Box Protein 7 (PAX-7)

PAX-7 is a highly conserved transcription factor shared by satellite cells of various species. The present invention targets for satellite cells and IPSC with porcine origin. Comparison of PAX-7 from different species showed an about 99% similarity of translated porcine PAX-7 polypeptides to human PAX-7 and an about 98% similarity to mouse PAX-7 (Ding, S., et al., 2017). Since no published data of translated porcine PAX-7 exists, the present invention utilizes human PAX-7.

The one letter code for human PAX-7 is shown below. Data was collected from uniprot.

>sp|P23759|PAX7_HUMAN Paired box protein Pax-7
OS = Homo sapiens OX = 9606 GN = PAX7 PE = 1
SV = 4
(SEQ ID NO: 2)
MAALPGTVPRMMRPAPGQNYPRTGFPLEVSTPLGQGRVNQLG
GVFINGRPLPNHIRHKIV
EMAHHGIRPCVISRQLRVSHGCVSKILCRYQETGSIRPGAIGGS
KPRQVATPDVEKKIEE
YKRENPGMFSWEIRDRLLKDGHCDRSTVPSGLVSSISRVLRIKF
GKKEEEDEADKKEDDG
EKKAKHSIDGILGDKGNRLDEGSDVESEPDLPLKRKQRRSRTTF
TAEQLEELEKAFERTH
YPDIYTREELAQRTKLTEARVQVWFSNRRARWRKQAGANQLA
AFNHLLPGGFPPTGMPTL
PPYQLPDSTYPTTTISQDGGSTVHRPQPLPPSTMHQGGLAAAA
AAADTSSAYGARHSFSS
YSDSFMNPAAPSNHMNPVSNGLSPQVMSILGNPSAVPPQPQAD
FSISPLHGGLDSATSIS
ASCSQRADSIKPGDSLPTSQAYCPPTYSTTGYSVDPVAGYQYG
QYGQTAVDYLAKNVSLS
TQRRMKLGEHSAVLGLLPVETGQAY

There is no need for any post-translational modification with PAX-7. However, in an embodiment, PAX-7 is modified with a cell penetrating peptide (CPP). The present invention utilizes the “trans-activator of transcription” (TAT) signal peptide. This cell-penetrating peptide can translocate numerous proteins, peptides, DNA, RNA, and small drugs into the cytoplasm with high efficiency. Moreover, this signal peptide, in an embodiment, is applied into the cell culture medium. When the TAT is applied, the cultured cells can absorb the transcription factor and the cells can be arrested in the proliferation phase, and differentiation can occur.

### Myogenin

Myogenin acts as a transcriptional activator that promotes transcription of muscle-specific target genes and plays a role in muscle differentiation, cell cycle exit, and muscle atrophy. Myogenin is essential for the development of functional embryonic skeletal fiber muscle differentiation.

The one letter code for porcine Myogenin is shown below. Data was collected from uniport.

>sp|P49812|MYOG_PIG Myogenin OS = Sus scrofa
OX = 9823 GN = MYOG PE = 3 SV = 1
(SEQ ID NO: 3)
MELYETSPYFYQEPHFYDGENYLPVHLQGFEPPGYERTELSLSPEARVPL
EDKGLGTPEH
CPGQCLPWACKVCKRKSVSVDRRRAATLREKRRLKKVNEAFEALKRST
LLNPNQRLPKVE
ILRSAIQYIERLQALLSSLNQEERDLRYRGGGGPQPGVPSECSSHSASCSP
EWGSALEFG
PNPGDHLLTADPTDAHNLHSLTSIVDSITVEDVAVAFPDETMPN

There is no need for any post-translational modification with Myogenin. However, myogenin needs to be modified with a CPP (cell penetrating peptide), and similar to PAX-7, the TAT signal peptide is used.

### Myogenic Differentiation 1 (MyoD1)

MyoD1 acts as a transcriptional activator that promotes transcription of muscle-specific target genes and plays a role in muscle differentiation. Together with MYF5 and MYOG, MyoD1 co-occupies the muscle specific gene promoter core region during myogenesis. Further, MyoD1 induces fibroblasts to differentiate into myoblasts.

The one letter code for porcine MyoD1 is shown below. Data was collected from uniport.

(SEQ ID NO: 4)
>sp|P49811|MYOD1_PIG Myoblast determination
protein 1 OS = Sus scrofa OX = 9823
GN = MYOD1 PE = 3 SV = 1
MELLSPPLRDVDLTGPDGSLCNFATADDFYDDPCFDSPDLRFFE
DLDPRLVHVGALLKPE
EHSHFPAAAHPAPGAREDEHVRAPSGHHQAGRCLLWACKACK
RKTTNADRRKAATMRERR
RLSKVNEAFETLKRCTSSNPNQRLPKVEILRNAIRYIEGLQALLR
DQDAAPPGAAAAFYA
PGPLPPGRGGEHYSGDSDASSPRSNCSDGMMDYSGPPSGARRR
NCYDGTYYSEAPSEPRP
GKNAAVSSLDCLSSIVESISTESPAAPALLLADTPRESSPGPQEAA
AGSEVERGTPTPSP DAAPQCPASANPNPIYQVL

In an embodiment, post-translational modification is necessary for MyoD1. MyoD1 is phosphorylated by CDK9 and this phosphorylation promotes its function in muscle differentiation. Further, MyoD1 is acetylated by a complex containing EP300 and PCAF. In an embodiment, the acetylation is essential to activate target genes.

Moreover, in an embodiment of the present invention, MyoD1 can be modified with a CPP. In one embodiment, the present invention contemplates use of the TAT signal peptide.

### Fat-Influencing Peptides

### Preadipocyte Factor 1 (Pref-1, Also Called Dlk1/FA1)

Pref-1 is a molecular gatekeeper of adipogenesis, which acts by maintaining the preadipocyte state and preventing adipocyte differentiation. The one letter code for porcine Pref-1 is shown below. Data was collected from uniport.

(SEQ ID NO: 5)
>tr|Q9GL46|Q9GL46_PIG Pref-1 (Fragment) OS = Sus
scrofa OX = 9823 PE = 4 SV = 1
GDFRCRCPAGFMDKTCSRPVSTCANEPCLNGGTCLQHSQVRFECLCKPKF
TGPLCGRKRA
AGPQQVTRLPSSYGLTYRLTPGVHELPVPQPEHRILKVSMKELTKSTPLL
SEGQAICFTI LGVLTSLVVLGTMGI

There is no need for any post-translational modification of Pref-1. However, Pref-1 may be modified with a CPP and the present invention contemplates use of the TAT signal peptide.

### Peroxisome Proliferator-Activated Receptor Gamma (PPAR-γ or PPARG)

PPARγ is a nuclear receptor that binds peroxisome proliferators, such as hypolipidemic drugs and fatty acids. Once activated by a ligand, the nuclear receptor binds to DNA specific PPAR response elements (PPRE) and modulates the transcription of its target genes, such as acyl-CoA oxidase. It therefore controls the peroxisomal beta-oxidation pathway of fatty acids. PPARγ is a key regulator of adipocyte differentiation and glucose homeostasis.

The one letter code for porcine PPARG is shown below. Data was collected from uniport.

(SEQ ID NO: 6)
>sp|O62807|PPARG_PIG Peroxisome proliferator-
activated receptor gamma OS = Sus scrofa OX = 9823
GN = PPARG PE = 2 SV = 1
MGETLGDSLIDPESDAFDTLSANISQEVTMVDTEMPFWPTNFGISSVDLS
VMDDHSHSFD
IKPFTTVDFSSISTPHYEDIPFPRADPMVADYKYDLKLQDYQSAIKVEPV
SPPYYSEKTQ
LYNKPHEEPSNSLMAIECRVCGDKASGFHYGVHACEGCKGFFRRTIRLKL
IYDRCDLNCR
IHKKSRNKCQYCRFQKCLAVGMSHNAIRFGRMPQAEKEKLLAEISSDIDQ
LNPESADLRA
LAKHLYDSYIKSFPLTKAKARAILTGKTTDKSPFVIYDMNSLMMGEDKIK
FKHITPLQEQ
SKEVAIRIFQGCQFRSVEAVQEITEYAKNIPGFVNLDLNDQVTLLKYGVH
EIIYTMLASL
MNKDGVLISEGQGFMTREFLKSLRKPFGDFMEPKFEFAVKFNALELDDSD
LAIFIAVIIL
SGDRPGLLNVKPIEDIQDNLLQALELQLKLNHPESSQLFAKLLQKMTDLR
HQIVTEVQLL QVIKKTETDMSLHPLLQEIYKDLY

There is no need for any post-translational modification of PPARγ. However, PPARγ may be modified with a CPP and the present invention contemplates use of the TAT signal peptide.

### CCAAT-Enhancer-Binding Proteins (or C/EBPs)

C/EBPs are a family of transcription factors composed of six members, named from C/EBPα to C/EBPζ. Specifically, C/EBPα is a transcription factor that coordinates proliferation arrest and the differentiation of myeloid progenitors, adipocytes, hepatocytes, and cells of the lung and the placenta. C/EBPα binds directly to the consensus DNA sequence 5′-T[TG]NNGNAA[TG]-3′, acting as an activator on distinct target genes (Pabst, T., et al., 2001). Further, C/EBPα plays essential and redundant functions with CEBPB during early embryogenesis. C/EPBα is necessary for terminal adipocyte differentiation.

The one letter code for human C/EBP-α is shown below. There is no published data of porcine origin. Data was collected from uniprot.

(SEQ ID NO: 7)
>sp|P49715|CEBPA_HUMAN CCAAT/enhancer-binding
protein alpha OS = Homo sapiens OX = 9606
GN = CEBPA PE = 1 SV = 3
MESADFYEAEPRPPMSSHLQSPPHAPSSAAFGFPRGAGPAQPPA
PPAAPEPLGGICEHET
SIDISAYIDPAAFNDEFLADLFQHSRQQEKAKAAVGPTGGGGG
GDFDYPGAPAGPGGAVM
PGGAHGPPPGYGCAAAGYLDGRLEPLYERVGAPALRPLVIKQE
PREEDEAKQLALAGLFP
YQPPPPPPPSHPHPHPPPAHLAAPHLQFQIAHCGQTTMHLQPGH
PTPPPTPVPSPHPAPA
LGAAGLPGPGSALKGLGAAHPDLRASGGSGAGKAKKSVDKNS
NEYRVRRERNNIAVRKSR
DKAKQRNVETQQKVLELTSDNDRLRKRVEQLSRELDTLRGIFR
QLPESSLVKAMGNCA

Post-translational modification of C/EBPα includes phosphorylation at Thr-226 and Thr-230 by GSK3, with the post-translation modification being constitutive in adipose tissue Further, in an embodiment, C/EBPα is modified with a CPP. In an embodiment, the present invention utilizes the TAT signal peptide as the peptide that modifies C/EBPα.

Supplemental Ingredients for Advanced Differentiation

In an embodiment, the present invention relates to adding supplemental ingredients to the other embodiments described herein. In an embodiment, it has been discovered that supplemental ingredients support the novel differentiation via a TAT-coupled transcription factor, as well as conventional differentiation without these factors. These ingredients range from, fatty acids, small molecules to other supplemental ingredients for differentiation.

### Supplemental Ingredients for Fat Differentiation

In an embodiment, unsaturated fatty acids, surfactants, lipids, polyphenols, and other small molecules can be added to the media of the present invention to aid in the differentiation of the adult stem cells. The following lists show the various and specific components that can be added.
Unsaturated FA: Erucic acid, Elaidic acid, Oleic Acid, Pahnitoleic acid, Myristoleic acid Phytanic acid, Pristanic acid, linoleic acid
Surfactants: albumins, putrescine, spermine

### Supplemental Ingredients for Muscle Differentiation

Lipids: Lysophosphatidic acid (LPA),
Polyphenols: Epicatechine
small molecules: Trichostatin A, PD 98,059

In an embodiment, the present invention relates to a fusion protein comprising at least one TF and at least one CPP. In an embodiment, the TF-CPP fusion protein may comprise a recombinant fusion protein, meaning that the fusion protein has been produced in a host cell that has been either transformed or transfected with a polynucleotide encoding the fusion protein or produces the fusion protein as a result of homologous or heterologous recombination. Other methods for producing the TF-CPP fusion protein, are contemplated and therefore within the scope of the present invention. These methods included but are not limited to methods such as by chemical cross-linking.

The TF-CPP fusion protein may also be prepared using techniques known in the art. In one method, a TF may be fused to a CPP using a suitable host, such as a eukaryotic or prokaryotic cell. For example, a cDNA encoding a TF-CPP fusion protein may be constructed to include nucleic acid sequences encoding both a TF and a CPP. The nucleic acid sequences may be in-frame and may be located downstream of an N-terminal leader sequence (e.g., a sequence comprising a 6-Histidine tag). The N-terminal leader sequence may enable purification of the expressed recombinant TF-CPP fusion protein using methods known in the art.

The fusion protein may contain a linker and optionally may contain a purification domain. The purification domain aids in purifying recombinantly made proteins. Methods of purification include the use of the 6-his tag alluded to above. Other methods include using affinity chromatography or other forms of chromatography. When using affinity chromatography, nickel or cobalt resins may be used to bind the fusion protein. Other methods such as the use of GST, attaching antibodies or small molecules to the fusion protein sequence are contemplated (such as using FLAG peptide, or biotin/streptavidin technologies). In a variation, the linker region may also serve as the purification region.

In an embodiment, recombinant techniques may be used to make the fusion protein. Generally, this involves removing the stop codon from the first protein/polypeptide gene sequence (e.g., the transcription factor) that is on the 5′ side of the fusion protein gene sequence and then optionally adding the linker gene sequence and the gene sequence from the other desired protein (e.g., the second protein or peptide sequence, which may be the CPP gene sequence) that is downstream (closer to the 3′ end) of the first protein gene sequence. In a variation, the entire gene sequences may not be used in the fusion protein gene sequence. In a variation, protein fragments of transcription factors and CPP may be used that have the requisite activity. The fusion protein gene sequence can then generate the fusion protein using recombinant technologies. The isolation and purification of the fusion protein takes place using technologies alluded to herein.

In an embodiment, the present invention relates to a method (of inducing differentiation of adult stem cells and/or undergoing a cell culture process) wherein said method comprises: utilizing a transcription factor to induce differentiation of adult stem cells or induced pluripotent stem cells (IPSC) into myocytes or adipocytes; and applying the myocytes and/or the adipocytes to a cell culture process via addition into a culture medium. In a variation, the adult stem cells comprise satellite cells and mesenchymal stem cells. In a variation, the transcription factor comprises a muscle-inducing peptide/protein or a fat-inducing peptide/protein. In a variation, the muscle-inducing peptide/protein is one or more members selected from the group consisting of: PAX-7, Myf5, MyoD1 and Myogenin, and the method further comprises: engaging in post-translational modification of the candidate.

In a variation, the fat-inducing peptide/protein is one or more members selected from the group consisting of: Pref-1, PPAR-γ, and C/EBPα and the method further comprises: engaging in post-translational modification of the candidate. In a variation, the method further comprises: modifying the muscle-inducing peptide/protein or the fat-inducing peptide/protein with a cell penetrating peptide. In a variation, the cell penetrating peptide comprises a trans-activator of transcription (TAT) signal peptide. In a variation, the muscle-inducing peptide/protein and/or the fat-inducing peptide/protein is transcribed and translated from a DNA fusion protein gene sequence that comprises a gene sequence for the muscle-inducing peptide/protein or the fat-inducing peptide/protein and a gene sequence for the cell penetrating peptide.

In an embodiment, the DNA fusion protein gene sequence further comprises a linker DNA sequence and/or a purifying peptide gene sequence. In a variation, the fusion protein gene sequence comprises a linker DNA sequence and a purifying peptide gene sequence. In a variation, the fusion protein gene sequence comprises one or more gene sequences selected from the group consisting of gene sequences that encode PAX-7, Myf5, MyoD1, Myogenin, Pref-1, PPAR-γ, C/EBPα, and TAT. In a variation, the purifying peptide gene sequence is a his tag.

In an embodiment, the method further comprises adding supplemental ingredients to the cell culture process. In a variation, the supplemental ingredients comprise one or more of unsaturated fatty acids, surfactants, lipids, polyphenols, or other small molecules. In a variation, the supplemental ingredients are one or more members selected from the group consisting of Erucic acid, Elaidic acid, Oleic Acid, Palmitoleic acid, Myristoleic acid, Phytanic acid, Pristanic acid, linoleic acid, albumins, putrescine, spermine, Lysophosphatidic acid (LPA), Epicatechine, Trichostatin A, and PD 98,059. In a variation, the myocytes and/or the adipocytes comprise a cell line that is used to create a cultured, clean meat, or in vitro meat product.

In an embodiment, the present invention relates to a meat product made by a method of utilizing a transcription factor to induce differentiation of induced pluripotent stem cells (IPSC) adult stem cells into myocytes or adipocytes; and applying the myocytes and/or the adipocytes to a cell culture process via addition into a culture medium, wherein the cell culture process is a process to generate a meat product. In a variation, the transcription factor is a fusion protein that further comprises a cell penetrating peptide, and optionally comprises a linker segment and a purification segment.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### REFERENCES

The following references, to the extent that they provide exemplary procedural or other details supplementary to those set forth herein, are specifically incorporated herein by reference.
Chung, S. S., et al. (2010). Control of adipogenesis by the SUMO-specific protease SENP2. Mol. Cell. Biol. DOI: 10.1128/MCB.00852-09.
Cooper, R. N., et al. (1999). In vivo satellite cell activation via Myf5 and MyoD in regenerating mouse skeletal muscle. J. Cell. Sci. PMID: 10444384.
Cornelison, D. D., et al. (2000). MyoD(−/−) satellite cells in single-fiber culture are differentiation defective and MRF4 deficient. Dev. Biol. DOI: 10.1006/dbio.2000.9682.
Cornelison, D. D. & Wold, B. J. (1997). Single-cell analysis of regulatory gene expression in quiescent and activated mouse skeletal muscle satellite cells. Dev. Biol. DOI: 10.1006/dbio.1997.8721.
Darnell Jr., J. E. (1982). Variety in the level of gene control in eukaryotic cells. Nature. DOI: 10.1038/297365a0.
Davis, R. L., Weintraub, H. & Lassar, A. B. (1987). Expression of a single transfected cDNA converts fibroblasts to myoblasts. Cell. DOI: 10.1016/0092-8674(87)90585-x.
Dezawa, M., et al. (2005). Bone marrow stromal cells generate muscle cells and repair muscle degeneration. Science. DOI: 10.1126/science.1110364.
Ding, S., et al. (2017). Characterization and isolation of highly purified porcine satellite cells. Cell Death Discov. DOI: 10.1038/cddiscovery.2017.3.
Fittipaldi, A. & Giacca, M. (2005). Transcellular protein transduction using the Tat protein of HIV-1. Adv. Drug Deliv. Rev. DOI: 10.1016/j.addr.2004.10.011.
Füchtbauer, E. & Westphal, H. (1992). MyoD and myogenin are coexpressed in regenerating skeletal muscle of the mouse. Dev. Dyn. DOI: 10.1002/aja.1001930106.
Green, M. & Loewenstein, P. M. (1988). Autonomous functional domains of chemically synthesized human immunodeficiency virus tat trans-activator protein. Cell. DOI: 10.1016/0092-8674(88)90262-0.
Grounds, et al. (1992). Identification of skeletal muscle precursor cells in vivo by use of MyoD1 and myogenin probes. Cell Tissue Res. DOI: 10.1007/BF00318695.
Hidema, S., et al. (2012). Effects of protein transduction with intact myogenic transcription factors tagged with HIV-1 Tat-PTD (T-PTD) on myogenic differentiation of mouse primary cells. J. Biosci. Bioeng. DOI: 10.1016/j.jbiosc.2011.08.025.
Ho, A., et al. (2001). Synthetic protein transduction domains: enhanced transduction potential in vitro and in vivo. Cancer Res. PMID: 11212234.
Hua, W., et al. (2013). Elevation of protein kinase Ca stimulates osteogenic differentiation of mesenchymal stem cells through the TAT-mediated protein transduction system. Biochem. Cell Biol. DOI: 10.1139/bcb-2013-0035.
Karn, J. (2011). The molecular biology of HIV latency: breaking and restoring the Tat-dependent transcriptional circuit. Curr. Opin. HIV AIDS. DOI:
Kashanchi, F., et al. (1996). Interaction of human immunodeficiency virus type 1 Tat with a unique site of TFIID inhibits negative cofactor Dr1 and stabilizes the TFIID-TFIIA complex. J. Virol. DOI: 10.1128/jvi.70.8.5503-5510.1996.
Latchman, D. S. (1993). Transcription factors: an overview. Int. J. Exp. Pathol. PMID: 8217775.
Lefterova, M. I., et al. (2008). PPARgamma and C/EBP factors orchestrate adipocyte biology via adjacent binding on a genome-wide scale. Genes Dev. DOI: 10.1101/gad.1709008.
Lowe, C. E., O'Rahilly, S., & Rochford, J. J. (2011). Adipogenesis at a glance. J. Cell. Sci. DOI: 10.1242/jcs.079699.
Mäe, M. & Langel, U. (2006). Cell-penetrating peptides as vectors for peptide, protein and oligonucleotide delivery. Curr. Opin. Pharmacol. DOI: 10.1016/j.coph.2006.04.004.
Mann, D. A. & Frankel, A. D. (1991). Endocytosis and targeting of exogenous HIV-1 Tat protein. EMBO J. PMID: 2050110.
Milletti, F. (2012). Cell-penetrating peptides: classes, origin, and current landscape. Drug Discov. Today. DOI: 10.1016/j.drudis.2012.03.002.
Noda, T., et al. (2009). Transduction of MyoD protein into myoblasts induces myogenic differentiation without addition of protein transduction domain. Biochem. Biophys. Res. Commun. DOI: 10.1016/j.bbrc.2009.03.060.
Pabst, T., et al. (2001). Dominant-negative mutations of CEBPA, encoding CCAAT/enhancer binding protein-alpha (C/EBPalpha), in acute myeloid leukemia. Nat. Genet. DOI: 10.1038/85820.
Patruno, M., et al. (2017). A mini-review of TAT-MyoD fused proteins: state of the art and problems to solve. Eur. J. Transl. Myol. DOI: 10.4081/ejtm.2017.6039.
Rantanen, J., et al. (1995). Satellite cell proliferation and the expression of myogenin and desmin in regenerating skeletal muscle: evidence for two different populations of satellite cells. Lab Invest. PMID: 7898053.
Rosen, E. D. & MacDougald, O. A. (2006). Adipocyte differentiation from the inside out. Nat. Rev. Mol. Cell Biol. DOI: 10.1038/nrm2066.
Schwarze, S. R., Hruska, K. A., & Dowdy, S. F. (2000). Protein transduction: unrestricted delivery into all cells? Trends Cell Biol. DOI: 10.1016/s0962-8924(00)01771-2.
Silhol, M., et al. (2002). Different Mechanisms for Cellular Internalization of the HIV-1 Tat-Derived Cell Penetrating Peptide and Recombinant Proteins Fused to Tat. European Journal of Biochemistry. DOI: 10.1046/j.0014-2956.2001.02671.x.
Smith, C. K., Janney, M. J., & Allen, R. E. (1994). Temporal expression of myogenic regulatory genes during activation, proliferation, and differentiation of rat skeletal muscle satellite cells. J. Cell. Physiol. DOI: 10.1002/jcp.1041590222.
Sung, M. S., et al. (2013). Efficient myogenic differentiation of human adipose-derived stem cells by the transduction of engineered MyoD protein. Biochem. Biophys. Res. Commun. DOI: 10.1016/j.bbrc.2013.06.058.
Uysal, C. & Mizuno, H. (2010). Tendon regeneration and repair with adipose derived stem cells. Curr. Stem. Cell Res. Ther. DOI: 10.2174/157488810791268609.
Vivès, E. Brodin, P. & Lebleu, B. (1997). A truncated HIV-1 Tat protein basic domain rapidly translocates through the plasma membrane and accumulates in the cell nucleus. J. Biol. Chem. DOI: 10.1074/jbc.272.25.16010.
Wadia, J. S. & Dowdy, S. F. (2005). Transmembrane delivery of protein and peptide drugs by TAT-mediated transduction in the treatment of cancer. Adv. Drug Deliv. Rev. DOI: 10.1016/j.addr.2004.10.005.
Wang, Y., Hudak, C., & Sul, H. S. (2010). Role of preadipocyte factor 1 in adipocyte differentiation. Clin. Lipidol. DOI: 10.2217/clp.09.80.
Wang, Y. & Sul, H. S. (2009). Pref-1 regulates mesenchymal cell commitment and differentiation through Sox9. Cell Metab. DOI: 10.1016/j.cmet.2009.01.013.
White, U. A. & Stephens, J. M. (2010). Transcriptional factors that promote formation of white adipose tissue. Mol. Cell. Endocrinol. DOI: 10.1016/j.mce.2009.08.023.
Yin, H., Price, F., & Rudnicki, M. A. (2013). Satellite cells and the muscle stem cell niche. Physiol. Rev. DOI: 10.1152/physrev.00043.2011.
Zou, L., et al. (2017). Progress in Research and Application of HIV-1 TAT-Derived Cell-Penetrating Peptide. J. Membr. Biol. DOI: 10.1007/s00232-016-9940-z.

What is claimed is:

1. A method comprising:
utilizing a transcription factor to induce differentiation of adult stem cells induced pluripotent stem cells (IPSC) into myocytes or adipocytes; and
applying the myocytes and/or the adipocytes to a cell culture process via addition into a culture medium.

2. The method of claim 1, wherein the adult stem cells comprise satellite cells and mesenchymal stem cells.

3. The method of claim 1, wherein the transcription factor comprises a muscle-inducing peptide/protein or a fat-inducing peptide/protein.

4. The method of claim 3, wherein the muscle-inducing peptide/protein is one or more members selected from the group consisting of: PAX-7, Myf5, MyoD1 and Myogenin.

5. The method of claim 3,
wherein the muscle-inducing peptide/protein comprises the MyoD1, and
wherein the method further comprises: engaging in post-translational modification of the MyoD1.

6. The method of claim 3, wherein the fat-inducing peptide/protein is one or more members selected from the group consisting of: Pref-1, PPAR-γ, and C/EBPα.

7. The method of claim 3,
wherein the fat-inducing peptide/protein comprises the C/EBPα, and
wherein the method further comprises: engaging in post-translational modification of the C/EBPα.

8. The method of claim 3, further comprising:
modifying the muscle-inducing peptide/protein or the fat-inducing peptide/protein with a cell penetrating peptide.

9. The method of claim 8, wherein the cell penetrating peptide comprises a trans-activator of transcription (TAT) signal peptide.

10. The method of claim 8, wherein the muscle-inducing peptide/protein and/or the fat-inducing peptide/protein is transcribed and translated from a DNA fusion protein gene sequence that comprises a gene sequence for the muscle-inducing peptide/protein or the fat-inducing peptide/protein and a gene sequence for the cell penetrating peptide.

11. The method of claim 10, wherein the DNA fusion protein gene sequence further comprises a linker DNA sequence and/or a purifying peptide gene sequence.

12. The method of claim 11, wherein the fusion protein gene sequence comprises a linker DNA sequence and a purifying peptide gene sequence.

13. The method of claim 12, wherein the fusion protein gene sequence comprises one or more gene sequences selected from the group consisting of gene sequences that encode PAX-7, Myf5, MyoD1, Myogenin, Pref-1, PPAR-γ, C/EBPα, and TAT.

14. The method of claim 12, wherein the purifying peptide gene sequence is a his tag.

15. The method of claim 3, wherein the method further comprises adding supplemental ingredients to the cell culture process.

16. The method of claim 15, wherein the supplemental ingredients comprise one or more of unsaturated fatty acids, surfactants, lipids, polyphenols, or other small molecules.

17. The method of claim 16, wherein the supplemental ingredients are one or more members selected from the group consisting of Erucic acid, Elaidic acid, Oleic Acid, Palmitoleic acid, Myristoleic acid, Phytanic acid, Pristanic acid, linoleic acid, albumins, putrescine, spermine, Lysophosphatidic acid (LPA), Epicatechine, Trichostatin A, and PD 98,059.

18. The method of claim 1, wherein the myocytes and/or the adipocytes comprise a cell line that is used to create a cultured, clean meat, or in vitro meat product.

19. A meat product made by a method of utilizing a transcription factor to induce differentiation of induced pluripotent stem cells (IPSC) adult stem cells into myocytes or adipocytes; and
applying the myocytes and/or the adipocytes to a cell culture process via addition into a culture medium, wherein the cell culture process is a process to generate a meat product.

20. The meat product of claim 19, wherein the transcription factor is a fusion protein that further comprises a cell penetrating peptide, and optionally comprises a linker segment and a purification segment.
